(19) United States
(12) Patent Application Publication
(10) Pub. No.: US 2011/0087077 A1
(43) Pub. Date: Apr. 14, 2011

(54) SYSTEM AND METHOD OF PROVIDING PATIENTS COST SAVING INCENTIVES FOR HEALTHY BEHAVIORS

(76) Inventor: Anup Mahesh Desai, Vallejo, CA (US)

(21) Appl. No.: 12/903,204

(22) Filed: Oct. 13, 2010

Related U.S. Application Data
(60) Provisional application No. 61/250,988, filed on Oct. 13, 2009.

(51) Int. Cl. A61B 5/00

(52) U.S. Cl. 600/300

(57) ABSTRACT

Methods for monitoring a patient's health are provided. The methods compromise the steps of providing a website accessible via a computer for monitoring a patient's health; entering the patient's clinical data onto the website; analyzing the data to assign at least one preventive care protocol to the patient; providing a clinic for monitoring the progress of the patient's health; and providing the patient at least one incentive should the patient's health improve.

### CROSS REFERENCE TO RELATED APPLICATION

This application is a non-provisional application of, and claims priority to, provisional application U.S. Ser. No. 61/250,988, filed on Oct. 13, 2009, the contents of which are herein incorporated by reference in their entirety.

### FIELD OF THE INVENTION

The present invention is directed to a system and method of monitoring and incentivising patient and community healthy behaviors and providing payors the resulting cost savings for determining appropriate incentives.

### BACKGROUND OF THE INVENTION

Healthcare costs in this country are primarily driven by poor lifestyle choices. Many attempts have been made to incentivise doctors to achieve quality and preventative healthcare goals in their patient populations to help decrease costs. However, this has often times failed. Therefore, there is a need for a healthy behavior monitoring and incentive system that may be carried out in any healthcare setting and that would ultimately reduce costs and improve the health of the patient and the community as a whole. Often times, patients do not have full access to and/or do not know how to improve their health. Thus, there is a need for a system that includes a centralized database of preventative care protocols that may be complementary to and/or integrated with an electronic medical record (EMR). This would allow, for example, patients to access their preventive care protocols and become more knowledgeable about their health.

### SUMMARY OF THE PREFERRED EMBODIMENTS

In a preferred embodiment, a method for monitoring a patient's health is provided. The method comprises the steps of providing a website accessible via a computer for monitoring a patient's health; entering the patient's clinical data onto the website; using the data to assign at least one preventive care protocol to the patient; providing a clinic for monitoring the progress of the patient's health; and providing the patient with at least one incentive should the patient's health improve. Preferably, the step of entering includes the step of entering the patient's contextual data onto the website. The website is accessible by one or more of the following: the patient; a payor; an analyst; and a health care provider. Preferably, the payor provides the incentive. The method may further comprise the step of providing a geographic information system application, wherein the geographic information system application provides an analyst with information regarding geographical trends in health care. The method may further comprise the step of integrating information entered onto the website onto an electronic medical records system. The method may also further comprise the steps of determining whether the patient complies with the preventive care program and determining cost savings derived from such compliance.

In a preferred embodiment, a system for monitoring a patient's health is provided. The system comprises an Internet website accessible via a computer for monitoring the patient's health; a database of patient information; a database of preventive care protocols; and a clinic for monitoring the progress of the patient's health. In one aspect of this embodiment, the system further comprises a database of payor costs/savings. Preferably, the system comprises a geographic information system application. Preferably, the system comprises software for determining appropriate preventive care protocols using the patient's information, and/or software to execute preventive care algorithms based upon patient information. In one aspect of this embodiment, the system may further comprise a database that may be integrated with an electronic medical records system and/or a social networking component.

In a preferred embodiment, a system for monitoring at least one patient's health is provided. The system comprises an Internet website accessible via a computer for monitoring the patient's health; a database of patient clinical and contextual information; a database of preventive care protocols; a means for assigning at least one preventive care protocol to the patient based upon patient clinical data; a clinic for monitoring the progress of the patient's health; a geographic information system application, wherein the geographic information system application organizes patient clinical and contextual data based upon geographic location of the patient. Preferably, the geographic information system creates one or more community profiles based upon patient clinical and contextual data. In one aspect of this embodiment, the community profiles are used to generate preventive care protocols for the community.

### BRIEF DESCRIPTION OF THE DRAWINGS

The invention may be more readily understood by referring to the accompanying drawing in which:

FIG. 1 is block diagram of a system of providing patients cost saving incentives for healthy behaviors in accordance with a preferred embodiment of the present invention;

FIG. 2 is a block diagram of a computer system that may be used for implementing the system of FIG. 1, configured in accordance with a preferred embodiment of the present invention;

FIG. 3 is a network diagram illustrating a network of computer systems that may be used for implementing the system and method of FIG. 1, configured in accordance with a preferred embodiment of the present invention; and

FIG. 4 is a block diagram showing patient and preventative care clinic workflows in accordance with a preferred embodiment of the present invention.

### DETAILED DESCRIPTION OF THE PREFERRED EMBODIMENTS

The present invention is generally directed to a system and method of monitoring and rewarding patient healthy behaviors. Preferably, the system and method of the present invention is facilitated by the use of an Internet website (as used herein, “website” and “Internet website”, and “Internet” interchangeable) accessible via a computer (or any other device capable of displaying an Internet website such as an iPhone, PDA, mobile device, etc.). “Internet website” may also refer to any other electronic medium capable of data transmission. Preferably, the Internet website displays a graphical user interface (GUI).

A graphical user interface (GUI) is a type of user interface that allows people to interact with programs in more ways than just typing. The user generally is able to access the GUI by entering a secure website on the Internet, or any other electronic medium. It is to be understood that the GUI may be accessed via any web-enabled device, such as a computer, iPhone, PDA, other mobile device, television set, and the like. Preferably, the GUI is displayed on an Internet website accessible via a computer (as used herein, “an Internet website accessible via a computer” may mean “an Internet website on a computer” [or any other web-enabled device]).

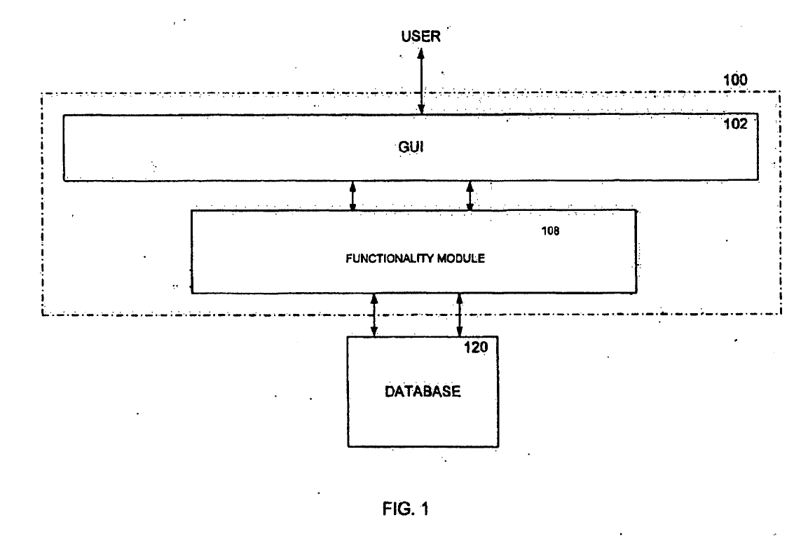

FIG. 1 is a block diagram of a system 100 of providing patients cost saving incentives for healthy behaviors in accordance with an embodiment of the invention. The system 100 includes a graphical user interface (GUI) 102 coupled to a functionality module 108. The GUI 102 provides a user with access to the information stored in a database 120 using the functionality provided by the functionality module 108. As further described below, in one embodiment, the database 120 stores information in one or more related databases that are logically grouped by predetermined areas. These areas may be, for example, patient information, preventive care protocols, cost savings information for payors, etc. As used herein, “database” should be understood to cover reference to a single database or a collection of databases. In other embodiments, information may be stored or arranged using a variety of data storage software and hardware techniques known to those of ordinary skill in the art in database programming. For example, one database system that may be used with the present invention is the SQL Server system as offered by Microsoft Corp. Other data storage systems, such as those based on Oracle Database products offered by Oracle Corporation or the MySQL database server offered by MySQL AB, may be used. These data storage systems run on a variety of hardware platforms, including those based on the microprocessors offered by Advanced Micro Devices2, Inc., or Intel Corp. In addition, the system may include a software system, for example, for selecting appropriate preventive care protocols based upon patient information.

Referring to FIG. 1, the database 120 is preferably accessible by the functionality provided by the functionality module 108 in the system 100. The user accesses the functionality module 108 through the GUI 102, as described herein. For example, the functionality module 108 may be a software system for identifying one or more preventive care protocols based upon patient clinical and/or contextual information. Subsequently, the selected preventive care protocol may be displayed on the GUI 102.

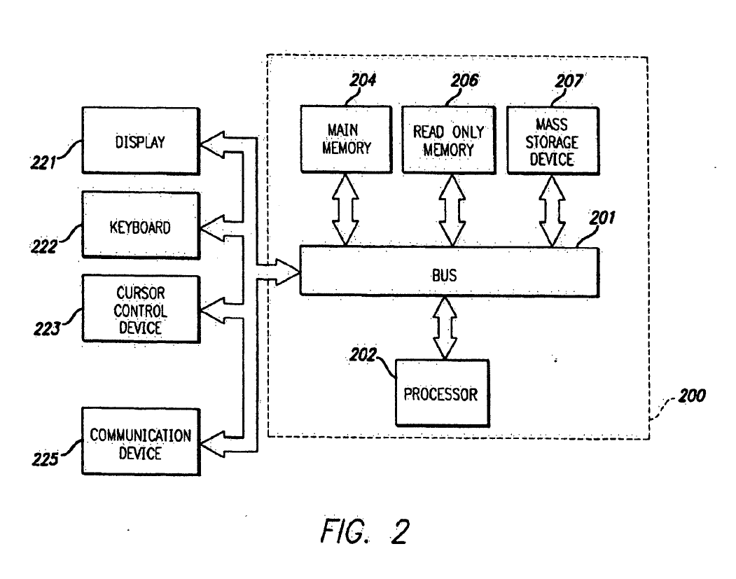

FIG. 2 illustrates an example of a computer system 200 in which the features of the present invention may be implemented. The computer system 200 includes a bus 201 for communicating information between the components in the computer system 200, and a processor 202 coupled with the bus 201 for executing software code, or instructions, and processing information. The computer system further comprises a main memory 204, which may be implemented using random access memory (RAM) and/or other random memory storage device, coupled to the bus 201 for storing information and instructions to be executed by the processor 202. The main memory 204 also may be used for storing temporary variables or other intermediate information during the execution of instructions by the processor 202. The computer system 200 also includes a read only memory (ROM) and/or other static storage device coupled to the bus 201 for storing static information and instructions for processor 202.

Another type of user input shown in the figure is a cursor control device 223, such as a conventional mouse, touch mouse, trackball, track pad, or other type of cursor direction keys for communicating direction information and command selection to the processor 202 and for controlling movement of a cursor on the display 221. Although not illustrated, the computer system 200 may optionally include video, camera, speakers, sound card, and many other conventional multimedia options. Various types of input devices, including, but not limited to the input devices described herein, unless otherwise noted, allow the user to provide command or input to the computer system 200. For example, in the various descriptions contained herein, reference may be made to the user “selecting,” “clicking,” or “inputting,” and any grammatical variations thereof, one or more items in a user interface. These should be understood to mean that the user is using one or more input devices to accomplish the input.

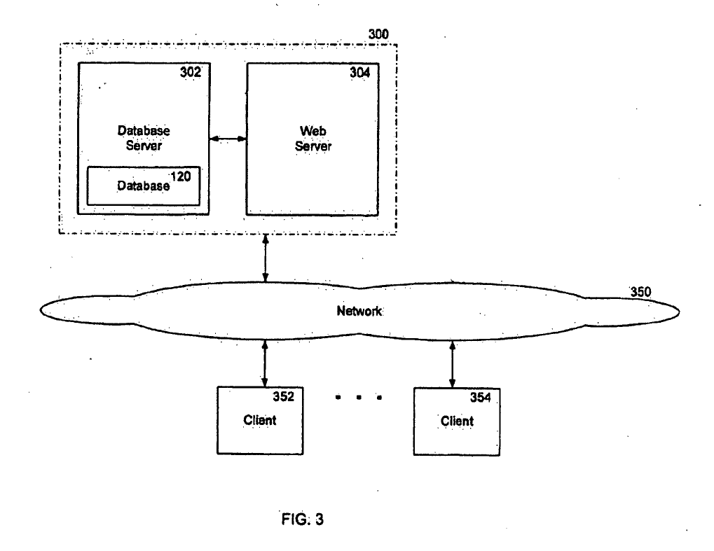

In accordance with one embodiment, FIG. 3 illustrates a computer network including a server system 300 having a database server 302 for storing a database 120 and a web server 304 that stores the instructions necessary to access and present the information from, as well as store information into, the database 120. The patient, using a client computer such as a client 352 and a client 354, accesses server system 300 through a network 350. Other systems, such as those belonging to a manufacturer may also be coupled to the server system 300. In the illustrated embodiment, the network 350 represents a variety of networks that may include one or more local area networks as well as wide area networks. The functionality provided by the database server 302, the web server 304, the client 352 and the client 354, as well as by the manufacturer may be implemented using a computer system having the characteristics of the computer system 200 described herein. It should be noted, however, that the specific implementation of the computer system or systems used to describe the present invention is not to be limiting unless otherwise specifically noted. For example, the functionality provided by the database server 302 and the web server 304 may be offered by one computer system. Further, the functionality provided by the database server 302 and 304 may be redistributed over several computers.

In a preferred embodiment, the patient may interact with the information stored in the database 120 through the GUI 102. In the following description, the GUI 102 is implemented using one or more web pages (which may be referred to herein as “pages,” “screens,” or “forms”) provided by the web server 304 accessible by the user using any Internet web browser software, such as the Internet Explorer browser provided by Microsoft Corp., on a client computer such as the client 352. In another embodiment, one or more custom software programs can be created to implement the system described herein. Of course, the web server 304 may itself have browser software installed on it so as to be accessed by the user. Further, throughout the description of the various embodiments of the invention herein, references are made to the user performing such actions as selecting buttons or clicking on, executing searches or updates on the database 120. In one preferred embodiment, these requests are generated by the user interacting with the browser. Preferably, the one or more web pages described herein may include a form interface (e.g., a drop down menu, a radio button selection, etc.). Once the user has provided such selection, the user may select a button or a link on the web page to request an update of the database 120 with the information. The browser will send the web server 304 a link that includes the information (i.e., the user's selection) being sent as well as the request to update the database 120.

In a preferred embodiment, the system and methods of the present invention may be accessed via the website through cloud computing (i.e., virtual server). For example, cloud computing may provide a secure server for a patient, payor, health care provider, analyst, physician, and the like web enabled GUI access via a virtual server.

Cloud computing may serve to store and operate all of the GUIs, software, and databases. In other embodiments, cloud computing may not be used. For example, the server may not be virtual. As such, the server may be a physical computer having Internet access.

In a preferred embodiment, a few different people and/or entities may be able to enter and/or access information on the Internet website using any computer, mobile device, or any other device capable of displaying an Internet website, or other electronic medium. For example, a health care provider, analyst, patient, payor, and the like may have access to the Internet website. Preferably, the Internet website is a secure website. Preferably, log in information for each individual and/or entity determines what information that individual is privy to. For example, the patient may have access to only his or her information; however, the health technician may have access to the health care information of a panel of patients. In this manner, the GUI for the Internet website may appear to the same for each type of individual/entity; however, depending on the individual's and/or entity's identity, he or she may have access to some information, but not all. In other embodiments, the GUI may be tailored to each type of individual, such as a health care provider vs. patient. It is to be understood that information may be entered, i.e., typed in, or “selected” from, for example, a drop-down menu, or the like. As such, as used herein “enter” may refer to “select” or the like. As used herein, “health care provider” may refer to physician, nurse practitioner, clinician, physician's assistant, nurse, medical assistant, and the like.

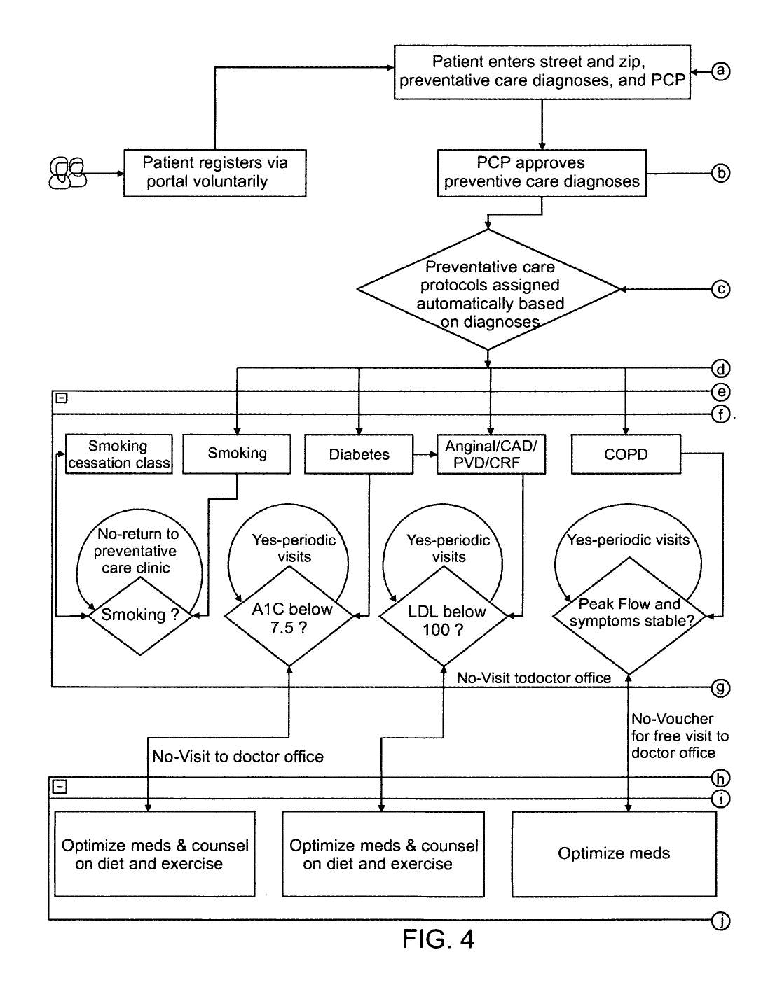

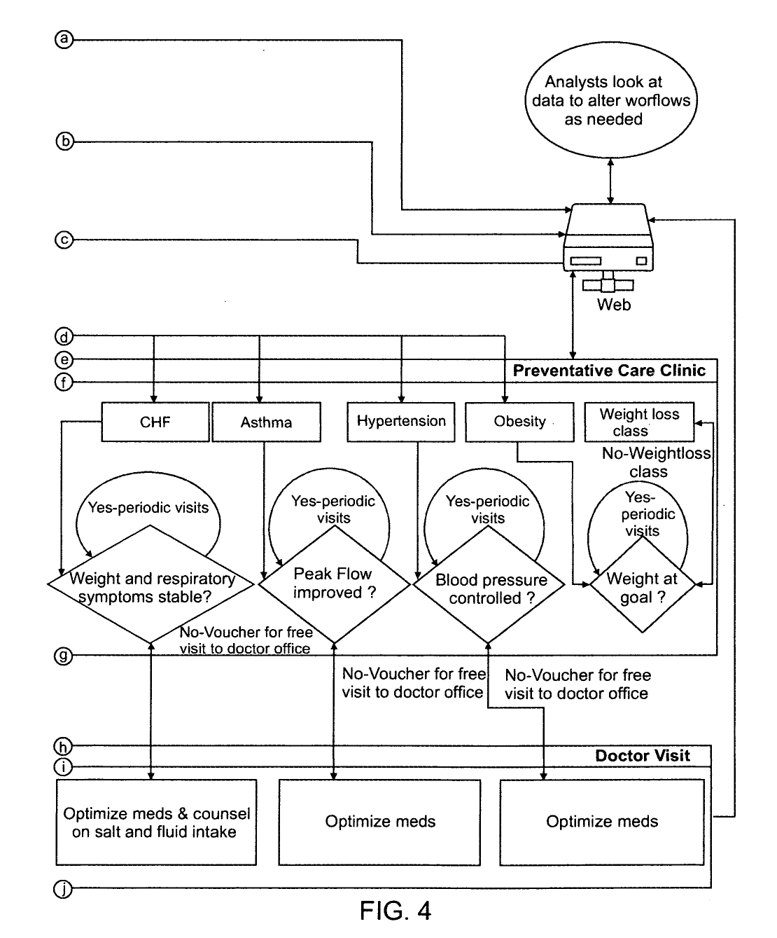

Referring to FIG. 4, in a preferred embodiment, a person voluntarily enters his or her information onto a server via the Internet using a GUI accessible via a computer, PC, mobile device such as an iPhone, and/or kiosk. For ease of description, these people will be referred to herein as a “patient.” Information would be stored in a database

In a preferred embodiment, the information that the patient enters is his or her clinical information (i.e., medical information, such as medical history, etc.) and contextual information (i.e., demographic, geographic, occupation and the like), as well as his or her clinician. As used herein. “clinical information” may refer to health information unique to the patient.

In a preferred embodiment, and as shown in FIG. 4, the health information is entered by selecting one or more illnesses, diseases, addictions, and/or conditions from a list of illnesses, diseases, addictions and/or conditions. In a highly preferred embodiment, theses illness, diseases, addictions, and/or conditions are preventable. This list of illnesses may include, but is not limited to, the following: heart attack, smoking, diabetes, coronary artery disease, chronic obstructive pulmonary disease, congestive heart failure, asthma, hypertension, heart attack, and obesity. In another embodiment, illnesses may be added and/or subtracted. In one aspect of this embodiment, the patient may be able to enter additional health information such as family history, social history, sexual history, medications, dates of past surgeries/procedures, and past MRI/CT/other imaging studies. In other embodiments, the patient does not enter his or her health information on the website, and the clinician or other health care provider may enter the information for the patient. In yet other embodiments, the patient and the health care provider both enter information into the website. In yet other embodiments, the patient is able to enter his or her preventive illness rather than selecting it from a preset list of checkable items. It is to be understood that the diseases/conditions, etc. do not need to be “preventable” to be monitored using the system and methods of the present invention.

In a preferred embodiment, the patient may be provided with a series of questions in order create his or her contextual profile. In this regard, preferably, the patient may be provided with a list of answers to these questions, and will be directed to choose from the list of answers. In other embodiments, the patient may be able to enter answers to the contextual questions by typing them in. Based on these answers, a contextual profile of the patient is established. Contextual profiles of patients residing in a particular geographic location may be grouped together to establish a community profile, as discussed herein. In other embodiments, contextual profiles may not be established for all or part of the participating patients.

In a preferred embodiment, and as shown in FIG. 4, at least one preventive care protocol is assigned to the patient based on the clinical information and/or the contextual information entered. Preferably, the preventive care protocol is assigned based on patient information entered, and confirmed by a clinical metric (e.g. blood pressure, weight, lab test result) and/or clinician validation.

In a preferred embodiment, the preventive care protocols are determined by software that routes patients to appropriate preventive care algorithms using information in the database (i.e., patient clinical and/or contextual information). As used herein, “patient information” may refer to patient clinical and/or contextual information. In other embodiments, the preventive care protocols may be established by physicians and/or other health care providers. In yet other embodiments, the preventive care protocols may be determined by both the clinician and the software algorithms. In other embodiments, the preventive care protocols may be designed for the community as a whole, depending, for example, on the community's health and/or demographics.

In a preferred embodiment, preventive care protocols may include one or more encounters with a preventive clinic and/or nutrition/lifestyle advice. As used herein, “preventive clinic,” “preventive care clinic” and “clinic” are interchangeable and refer to a physical clinic (i.e., an in-person clinic) or a “virtual” one, i.e., one in where the encounters are on-line, via web-cam. In a preferred embodiment, preventive care protocols include a scheduled series of encounters at a preventative clinic with a health provider for, for example, education on desired clinical goals and a preferred timeline for improvement. The clinic may be anywhere. As used herein, the preventive clinic may be an office at the patient's employer, school, or the like. The clinic may also be a location that the patient travels to, or it may be, for example, a technician that travels to the patient without departing from the scope of the present invention.

In a preferred embodiment, preventive care protocols may be modified/replaced depending on the patient's clinical and contextual information at the time. For example, if the patient's health improves, preventive care protocols may be modified accordingly. In this regard, the patient's health care provider, such as his or her physician, may review the patient's progress, and may modify the preventive care protocols via logging into a secure website accessible via a computer. For example, the physician and/or clinician may able to add any additional data/protocols that would ultimately improve preventive care outcomes. As such, subsequent protocols may include a series of patient encounters (virtual or in-person at the preventive care clinic) and may focus around maintaining goals achieved. For those initially not achieving clinical goal(s) within a set timeline, a protocol may be implemented for continued behavior education at the preventive clinic. For patients whose health improves, as well as patients whose health stays the same or declines, other protocols may include an on-line education regimen and/or added direction to seek clinician assistance for medical therapy optimization (i.e., changing dose of diabetes medication). It is to be understood that more than one preventive care protocol may be assigned to the patient at any given time. In other embodiments, physicians may receive only paper/email reports of patient progress. In yet other embodiments, physicians would have no access to patient preventive care outcomes/protocol data. In yet other embodiments, patient data may be interfaced to physician electronic medical records (EMR) to download/upload patient data and to change individual protocols as needed.

In a preferred embodiment, the preventive care protocols may be available for patient viewing and monitoring of achievements on the website accessible via a computer or other device.

In a preferred embodiment, the preventive care protocols may provide a timeline of when the patient should encounter his or her health care provider, depending on the patient's health/clinical information. Additionally, the patient may be notified of the need for encountering his or her health provider via an electronic notification, such as email, or a message when he or she logs on to the GUI accessible via an Internet website. For example, the patient may have predetermined encounters established by his or her protocol, and may have notifications for follow-up appointments sent out via telephone or electronic messages accessible through the patient's GUI. The patient may also be directed to follow-up with his/or her clinician to optimize current medications and/or to start new medications. Preferably, a patient failing to achieve these goals within, for example, a period of time such as 9 months, would then meet with the health care provider, such as the physician, for medication, behavior modification, and extensive behavior education at the preventive clinic or via on-line patient education.

In a preferred embodiment, the patient may encounter with the preventive care clinic at prescribed time intervals, depending at least in part on the patient's health. For example, a patient with diabetes may return for an A1C blood test every 3 months until the patient is able to consistently obtain A1C levels of less than 7.0% with lifestyle and medication changes. At this point, the frequency of A1C measurements (and thus encounters with the preventive care clinic) may be reduced to every 6 months. As an additional example, obese patients may have their height and weight taken for the purpose of calculating the body mass index every 6 months to monitor the progress of their lifestyle changes. As another example, a diabetic patient may have blood drawn at the preventive clinic to measure his or her A1C and LDL cholesterol, at which time the patient would provide the health care provider with a list of his or her prescribed diabetes medications, if any. The patient may subsequently return to the preventive clinic to review these results with the health technician and be provided behavior education and a protocol advising to return for repeat A1c test and LDL cholesterol testing every 3 months until the A1C is below 7.0% and the LDL cholesterol is below 100 mg/dL, for example. At each of these encounters with the preventive care clinic, healthy behaviors may be reinforced with patient education by a health care provider.

In a preferred embodiment, the health care provider located at the preventive care clinic is able to access all or part of the system to log patient data and/or achievements and provide appropriate behavioral education to improve patient outcomes at an individual level.

In a preferred embodiment, the health care provider and/or educator educates the patient regarding the disease and the goals associated therewith. For example, a patient with hypertension may be counseled on the importance of medication compliance and may be advised to be aware of lifestyle choices (e.g. salt intake, weight gain, caffeine and tobacco intake) that could prevent the patient from achieving the blood pressure goal. Patients that were not at goal within a few weeks, for example, may be directed to see their doctor for medication changes and evaluation of secondary causes of hypertension (e.g. sleep apnea, hyperthyroidism).

In a preferred embodiment, the patient receives “incentives” for every goal met, or met in part. Preferably, the health care provider determines which goal or part of a goal should receive an incentive. As used herein, “milestone” or “milestones” refer to these goals, or part of goals that should receive incentives. In this regard, the health care provider may input milestones into the database. The milestones may vary by individual and/or by community, or the like. In other embodiments, once the patient's clinical information is entered, then the milestones may be identified automatically. As such, they may be “pre-stored” into the database. Preferably, the network of vendors and specific products sold by these vendors eligible for voucher re-imbursement would be determined in advance. Preferably, the form/type of incentives is determined in advance; however, they may be determined at the time the milestone is achieved, or any other time. In yet other embodiments, patients do not receive incentives for goals met. In yet other embodiments, the network of vendors and/or specific products sold by these vendors eligible for voucher reimbursement would not be determined in advance (i.e., may be determined at the time each milestone is achieved).

In a preferred embodiment, incentives may be provided to the patient and/or the health care provider in electronic or paper form. For example, patients may be able to access their incentives via the website, and/or obtain their vouchers in paper form when they meet their health care provider in person at the preventive care clinic. Preferably, these incentives are vouchers for products and/or services that do not undermine the lifestyle changes of the patient. For example, an obese patient would not be given vouchers for calorie-dense fast food restaurants. In another preferred embodiment, patients would have some freedom to choose from a group of possible vouchers or incentives and/or suggest their own. Preferably, patients are able to log on to the website and see their incentive reimbursement history.

In a preferred embodiment, the patient may be able to interface with a social networking system (such as, but not limited to, a chat and/or discussion group) via a secure patient website accessible via a computer that would bring similar patients together to discuss health care concerns/treatments/lifestyle changes on-line. For example, patients with adult-onset diabetes may be grouped together in the database. In this regard, after the patient logs into the website, he or she will be given the choice of selecting the social networking system of the patient GUI. Preferably, the patient would be given the option of choosing one or more social networking groups based upon the clinical or contextual information entered. It is to be understood, however, that the social networking feature does not need to be provided.

In a preferred embodiment, the social networking system may enable patients to organize for on-line or in-person events to collectively improve their health. For example, patients may organize politically to influence community planning at the governmental level in order to encourage healthy lifestyles. For example, patients in support of setting up an exercise park/gym in a specific geographical area or in support of zoning for less fast food eateries in a certain geographical area may organize via the social networking system to influence local government. It is to be understand, however, the social networking system does not necessarily have to enable patients to organize collectively.

In preferred embodiment, the website may include a community map submenu displaying local healthcare events. Preferably, patients are able to geotag events to community map, which may provide a link to full event descriptions in the website for full details. In other embodiments, patients are not able to geotag events to the map and/or the community map does not exist.

In a preferred embodiment, a payor controls the amount, type, and timing of incentive payments. As used herein, “payor” refers to third parties subsidizing all or a portion of health care costs (i.e., employers). Preferably, the payor would be able to access a server via the Internet accessible via a computer to calculate the best amount, type, and timing of incentives to optimize preventive care outcomes. Preferably, such a system to calculate savings would be based on patient demographics (possibly including geographic location, and income), historical cost effectiveness data, real-time cost effectiveness data, and other data inputs. The final result of this determination may show up as attainable incentives for meeting preventive care goals. Preferably, the one or more payors may be able to modify the system to determine incentives. In other embodiments, amount, type, and/or timing of incentives may be determined by the health care provider or any other person. In yet other embodiments, any other person may be able to modify the system to determine incentives.

In a preferred embodiment, patient cost savings to payors may be calculated using software. These cost savings may be translated to patients in the form of rebate vouchers or other incentives.

In a preferred embodiment, one or more analysts would access the website accessible via a computer to analyze patient data to study effectiveness of current protocols and to make improvements/modifications to the protocols for specified groups of patients. For example, the analyst would log on and study the diabetes outcomes of patients in the community circumscribed by a particular zip code. The patients with A1c numbers above 7.0% might be retrieved by the analyst and presented in a geographic format with the ability to overlay demographic, medical, and contextual statistics. The website would also allow data viewing in traditional formats of graphs and charts to understand trends in the community. Preferably, based on this information, the analyst may test hypothesis for new patient community outreach health programs/incentives, or formulate new models of understanding disease processes. The analyst may also deploy surveys to patient participants in the social networking/discussion groups to include the patient perspectives on new incentives in order to improve community health. Such initiatives may then be advertised through the on-line patient site and through the preventive care clinics. Additionally, the website may permit communication with health care providers in the field to query their perspective on program implementation and outcomes.

In a preferred embodiment, a geographic information system application (referred to herein as “GIS”) is provided as part of the system of the present invention. A GIS application is a set of tools (i.e., software, databases, and the like) that captures, analyzes, manages, and presents data as it relates to geographic location. In other embodiments, the GIS application is not provided as part of the system.

In a preferred embodiment, such a GIS would incorporate patient data as well as other contextual data (e.g. crime statistics). Preferably, patients may voluntarily choose to have their clinical and/or contextual information incorporated into the GIS. Preferably, the one or more analysts may retrieve data from the GIS application in order to evaluate protocol effectiveness, as well as for planning patient and community programs and/or other additions/modifications that may enhance protocol effectiveness. For example, an analyst might look at a graphical format of a community in the form of a map and overlay demographics (e.g. crime statistics) based on zip code and also overlay health data (e.g. a1c) to evaluate community health trends. Based on this information, the analyst may formulate interventions for community health outreach programs. In this manner, the patient profiles may be complied and organized into community profiles. As such, preventive care protocols may be determined/modified based upon the community as a whole. Politicians and/or community leaders may be able to determine the need for a new gym, park, community health programs, and the like based upon these community profiles. Improvement of the community health may allow for certain tax benefits.

The following example is provided to illustrate the invention, but is not intended to limit the scope of the invention in any way.

Example of System and Methods

A patient is provided with log in information. The patient may have the option of determining his/her own password. The patient logs into the system using a website accessible via a computer. The patient subsequently enters his/her clinical and/or contextual information into the system. Health care providers, physicians, payors, and analysts may also be able to access all or part of the system, keeping in mind patient confidentiality issues. Data entered by the patient and/or health care provider is used to assign at least one preventive care protocol to the patient. This preventive care protocol may be selected from a list, or may be uniquely crafted for the patient and/or his community by software algorithms and/or the health care provider. Subsequently, the physician and/or clinician may modify the one or more preventive care protocols assigned to the patient. The patient may be provided health education at the preventive care clinic, depending on his or health condition. The requirement for physician follow-up may be determined base upon whether the patient has maintained and/or improved his or her health. If the patient has maintained or improved his or her health, the information is entered into the website, and the patient may be scheduled for another encounter with the preventive care clinic. If the patient has not maintained or improved his or her health, he or she is advised to see the physician. Should the patient maintain and/or improve is or her health, the patient is provided with at least one incentive. For example, the patient may be offered a free gym membership.

What is claimed is:

1. A method for monitoring at least one patient's health, the method comprising:
a. providing a website accessible via a computer for monitoring the patient's health;
b. entering the patient's clinical data onto the website;
c. using the data to assign at least one preventive care protocol to the patient;
d. providing a clinic for monitoring the progress of the patient's health; and
e. providing the patient at least one incentive should the patient's health improve.

2. The method of claim 1, wherein the step of entering includes the step of entering the patient's contextual data onto the website.

3. The method of claim 1, wherein the payor provides the incentive in step 1e.

4. The method of claim 2, further comprising the step of providing a geographical information system application, wherein the geographical information system application provides an analyst with information regarding geographical trends in health care.

5. The method of claim 1, further comprising the step of integrating information entered onto the website onto an electronic medical records system.

6. The method of claim 2, further comprising the step of providing health education to the patient at the clinic.

7. The method of claim 2, further comprising the step of providing a means for entering the patient's progress on the website.

8. The method of claim 2, wherein the preventive protocols are selected from the group comprising smoking, diabetes, coronary artery disease, chronic obstructive pulmonary disease, congestive heart failure, asthma, hypertension, and obesity.

9. The method of claim 8, further comprising the steps of determining whether the patient complies with the preventive care program and determining cost savings derived from such compliance.

10. The method of claim 9, wherein the costs savings are communicated to the payor.

11. The method of claim 4, wherein the geographical information system application is used to tailor the preventive care protocols.

12. A system for monitoring at least one patient's health, comprising:
a. an Internet website accessible via a computer for monitoring a patient's health;
b. a database of patient information;
c. a database of preventive care protocols; and
d. a clinic for monitoring the progress of the patient's health.

13. The system of claim 12, further comprising a database of payor costs/savings.

14. The system of claim 12, further comprising a geographic information system application.

15. The system of claim 12, further comprising a software system to execute preventive care algorithms based upon patient information.

16. The system of claim 12, further comprising a means for determining cost savings to the payor.

17. The system of claim 2, wherein the system further comprises a software program for calculating rebate totals and providing patients with vouchers for goods and services based on these totals.

18. The system of claim 12, wherein the patient information comprises clinical and contextual information.

19. A system for monitoring at least one patient's health, comprising:
a. an Internet website accessible via a computer for monitoring the patient's health;
b. a database of patient clinical and contextual information;
c. a database of preventive care protocols;
d. a means for assigning at least one preventive care protocol to the patient based upon patient clinical data;
e. a clinic for monitoring the progress of the patient's health;
f. a geographic information system application, wherein the geographic information system application organizes patient clinical and contextual data based upon geographic location of the patient;
wherein the geographic information system creates one or more community profiles based upon patient clinical and contextual data.

20. The system of claim 19, wherein the community profiles are used to generate preventive care protocols for the community.
